(19) United States
(12) Patent Application Publication
(10) Pub. No.: US 2022/0125715 A1
(43) Pub. Date: Apr. 28, 2022

(54) COMPARTMENTALIZED DRUG DELIVERY DEVICES

(71) Applicant: Particle Sciences Inc., Bethlehem, PA (US)

(72) Inventors: Jason L. McConnell, Bethlehem, PA (US); Mark A. Mitchnick, East Hampton, NY (US); Bruce L. Frank, Riegelsville, PA (US); Onajite Okoh, Philadelphia, PA (US)

(73) Assignee: Particle Sciences Inc., Bethlehem, PA (US)

(21) Appl. No.: 17/430,521

(22) PCT Filed: Feb. 14, 2020
(86) PCT No.: PCT/US2020/018326

§ 371 Date: Aug. 12, 2021

Related U.S. Application Data
(60) Provisional application No. 62/807,336, filed on Feb. 19, 2019.

(51) Int. Cl. A61K 9/00; A61K 47/34

(52) U.S. Cl. CPC A61K 9/0036; A61K 47/34; A61K 9/0092

(57) ABSTRACT

A delivery device for active pharmaceutical agents and made up of a hollow polymeric outer shape forming at least one closed internal cavity or compartment and containing a solid core of one or more active pharmaceutical agents and one or more excipients substantially unattached to the hollow polymeric outer shape is provided. Also provided are methods for production and use of this device.

This patent application claims the benefit of priority from International Application No. PCT/US2020/018326, filed Feb. 14, 2020, and U.S. Provisional Patent Application Ser. No. 62/807,336 filed Feb. 19, 2019, from which the PCT application claims priority, the disclosures of which are incorporated by reference in their entireties.

### FIELD

The present invention relates to delivery devices for active pharmaceutical agents and methods for their production and use. The delivery devices are made up of a hollow polymeric outer shell forming one or more closed internal cavities or compartments which contain one or more solid cores comprising one or more active pharmaceutical agents wherein the one or more solid cores are substantially unattached from the hollow polymeric outer shape thus forming an interspatial gap between the hollow polymeric outer shell and the solid core of the drug delivery device.

### BACKGROUND

Polymers have played an important role in drug delivery technology providing for controlled release of active pharmaceutical agents in constant doses over long periods of time, cyclic dosage and tunable release of both hydrophilic and hydrophobic drugs (Liechty et al. Annu. Rev. Chem. Biomol. Eng. 2010 1:149-173).

U.S. Pat. No. 8,343,528 discloses a drug delivery device for releasing one or more drugs at controlled rates for an extended period of time which comprises a reservoir comprising at least one active ingredient and optionally at least one pharmaceutically acceptable carrier, and a polyurethane based polymer completely surrounding the reservoir.

Published U.S. Patent Application No. 2014/0209100 discloses an intravaginal drug delivery device including a reservoir of at least one vaginally administrable drug wherein the reservoir is surrounded at least in part by a hydrophilic elastomer.

In certain instances, drug concentrations higher than the saturation solubility of a drug in a polymer may be desirable to achieve a target release rate. However, inclusion of high drug concentrations in a polymer drug delivery device can lead to migration of the drug to the surface of the device as it precipitates out of the solid solution. Such migration can cause an unwanted drug burst and/or drug actually blooming out of the device and forming a free drug coating on the device surface. Additionally, even when below this saturation point, a burst of drug release is often seen at early time-points following administration. In some cases, this burst is considered undesirable.

### SUMMARY

An aspect of the present invention relates to a delivery device for one or more active pharmaceutical agents. The device comprises a hollow polymeric outer shell forming at least one closed internal cavity or compartment. The device further comprises at least one solid core comprising one or more active pharmaceutical agents and one or more excipients inside the closed internal cavity or compartment and is substantially unattached from the hollow polymeric outer shape thus forming an interspatial gap between the hollow polymeric outer shell and the solid core of the drug delivery device.

Another aspect of the present invention relates to a method for production of a drug delivery device. The method comprises forming a hollow polymeric outer shell having at least one closed internal cavity or compartment. The method further comprises inserting at least one solid core comprising one or more active pharmaceutical agents and one or more excipients into the closed internal cavity or compartment while maintaining an interspatial gap between the hollow polymeric outer shell and the solid core of the drug delivery device and forming the drug delivery device from the filled hollow polymeric outer shell and at least one solid core.

Yet another aspect of the present invention relates to a method for delivering one or more active pharmaceutical agents to an individual in need thereof via the drug delivery device of the present invention.

### BRIEF DESCRIPTION OF THE FIGURES

FIGS. 1A, 1B and 10 are diagrams of a nonlimiting embodiment of the drug delivery device of the present invention wherein the hollow polymer shell is shaped as a vaginal ring and has a single compartment. The device prior to bonding into a ring (FIG. 1A), a cross section of the ring (FIG. 1B), and an inner view of the complete ring (FIG. 10) are shown.

FIGS. 2A, 2B and 2C are diagrams of a nonlimiting embodiment of the drug delivery device of the present invention wherein the hollow polymer shell is shaped as a vaginal ring and has multiple compartments. The device prior to bonding into a ring (FIG. 2A), a cross section of a ring (FIG. 2B), and an inner view of a complete ring (FIG. 10) are shown.

FIG. 3 is a graph showing daily progesterone release from various nonlimiting embodiments of drug delivery devices of the present invention shaped as vaginal rings.

FIG. 4 is a graph showing cumulative progesterone release from various nonlimiting embodiments of drug delivery devices of the present invention shaped as vaginal rings.

FIG. 5 shows the results from experiments comparing changes in surface area of the solid drug contained core on drug release from formulated compartmentalized devices of the present invention.

FIG. 6 shows results from experiments comparing changes in surface area of the hollow polymer shell on the daily release of progesterone from formulated compartmentalized devices of the present invention.

FIG. 7 shows the release of the drug progesterone from a monolithic (Matrix) vaginal ring and a compartmentalized vaginal ring prepared in accordance with the present invention. Both rings were made with the same polymers and drug.

FIG. 8 is a photograph comparing a compartmentalized device of the present invention (left) and a conventional core-sheath device (right) made with the same polymers and drug and aged for 14 months protected from light and moisture at ambient temperatures. In this comparison, the compartmentalized device of the present invention is made of 60% progesterone in a TPU28 rod insert inside a MPD-447i5 hollow polymer shell (left). The core-sheath device is made with 25% progesterone in TPU28 core and a MPD-447i5 sheath (right). Both devices were stored at ambient temperatures for 14 months.

### DETAILED DESCRIPTION

Drug delivery devices provided are designed to eliminate or significantly reduce both the burst release and surface migration of active pharmaceutical ingredients in the drug delivery devices.

The drug delivery devices of the present invention comprise a hollow polymeric outer shell having at least one closed internal cavity or compartment. The polymeric outer shell can be, among others, a tube or cylinder, the salient feature being that the outer shell of the device is continuous forming one or more closed internal cavities. Nonlimiting examples of shapes of the outer shell include vaginal rings, rods for subcutaneous implants and drug eluting films or patches. The polymeric outer shells have an inner and outer surface and a wall thickness ranging from about 150 um to about 750 um and an outer diameter ranging from about 1 mm to about 9 mm. However, as will be understood by the skilled artisan upon reading this disclosure, modifications can be made to the wall thickness as well as the outer diameter to manipulate active pharmaceutical ingredient (API) release.

Any biocompatible polymer can be used to produce the hollow polymeric outer shell. In one nonlimiting embodiment, the polymer is extrudable. In one nonlimiting embodiment, the polymer is hydrophilic. Preferred are polymers with water or media absorption of about 30% to about 100%, more preferable 35% to 100% including polymers with about 60% water/media absorption. In one nonlimiting embodiment, the polymer exhibits a hardness ranging from about 70 A to 100 A. In one nonlimiting embodiment, the polymer exhibits a hardness ranging from about 72 A to 95 A. Nonlimiting examples of polymers include polyurethanes, silicones, polyesters, polyolefins and copolymers thereof. In one nonlimiting embodiment, the polymer is a copolymer comprising ethylene vinyl acetate and poly(lactic-co-glycolic acid).

In some nonlimiting embodiments, the polymeric outer shell further comprises non-blooming concentrations of one or more active pharmaceutical ingredients.

The drug delivery devices of the present invention further comprise one or more solid cores comprising one or more active pharmaceutical agents and one or more excipients. In one nonlimiting embodiment, the solid core comprises a high concentration of one or more active pharmaceutical ingredients.

For purposes of the present invention, by “high concentration of one or more active pharmaceutical ingredients” it is meant a concentration above 20%. In one nonlimiting embodiment, the concentration ranges from about 20 to about 80%. In one nonlimiting embodiment, the concentration ranges from about 40% to about 60%.

Nonlimiting examples of excipients include polymers or other excipients capable of forming a solid core such as fillers such as sugars, including glucose, fructose, lactose, sucrose, mannitol, sorbitol, stevia extract, or sucralose; cellulose preparations such as, for example, maize starch, wheat starch, rice starch, potato starch, gelatin, gum tragacanth, methylcellulose, microcrystalline cellulose, hydroxypropyl methylcellulose, sodium carboxymethylcellulose; or others such as: polyvinylpyrrolidone (PVP or povidone) or calcium phosphate.

Any biocompatible polymer can be used as an excipient to produce the solid core. In one nonlimiting embodiment, the polymer is extrudable. In one nonlimiting embodiment, the polymer is hydrophilic. Preferred are polymers with water or media absorption of about 30% to about 100%, more preferable 35% to 100% including polymers with about 60% water/media absorption. In one nonlimiting embodiment, the polymer exhibits a hardness ranging from about 70 A to 100 A. In one nonlimiting embodiment, the polymer exhibits a hardness ranging from about 72 A to 95 A. Nonlimiting examples of polymers include polyurethanes, silicones, polyesters, polyolefins and copolymers thereof. In one nonlimiting embodiment, the polymer is a copolymer comprising ethylene vinyl acetate and poly(lactic-co-glycolic acid).

The solid core is sized to fit inside the closed internal cavity or compartment of the hollow polymeric outer shell and is substantially unattached from the hollow polymeric outer shell so that an interspatial gap is formed between the hollow polymeric outer shell and the solid core of the drug delivery device.

The interspatial gap between the hollow shell and the solid core may be empty or contain an agent such as, but not limited to, an osmotic agent such as sodium chloride to promote transfer of a biological fluid into the gap.

The core(s) contained within the compartment of the polymeric hollow shell may contain one or more active pharmaceutical ingredients. If two or more active pharmaceutical ingredients are used, the active pharmaceutical ingredients may be in the same solid core or different core in the same shell. The shell may have a single compartment, two compartments or multiple compartments each holding one or more solid cores.

Any active pharmaceutical ingredient deliverable via a polymeric drug delivery device can be incorporated into and delivered to an individual in need via the devices of the present invention. Nonlimiting examples include drugs, including vaccines, nutritional agents, cosmeceuticals and diagnostic agents. Examples of active pharmaceutical ingredients for use in the present invention include, but are not limited to analgesics, anti-anginal agents, anti-arrhythmic agents, anti-angiogenic agents, antibacterial agents, anti-benign prostate hypertrophy agents, anti-coagulants, anti-depressants, anti-diabetic agents, anti-epileptic agents, anti-fungal agents, anti-gout agents, anti-hypertensive agents, anti-inflammatory agents, anti-malarial agents, anti-migraine agents, anti-muscarinic agents, anti-neoplastic agents, anti-obesity agents, anti-osteoporosis agents, anti-parkinsonian agents, anti-protozoal agents, anti-thyroid agents, anti-urinary incontinence agents, anti-viral agents, anxiolytics, beta-blockers, cardiac inotropic agents, cognition enhancers, corticosteroids, COX-2 inhibitors, diuretics, erectile dysfunction improvement agents, essential fatty acids, gastrointestinal agents, histamine receptor antagonists, hormones, immunosuppressants, keratolyptics, leukotriene antagonists, lipid regulating agents, macrolides, muscle relaxants, non-essential fatty acids, nutritional agents, nutritional oils, protease inhibitors and stimulants.

Various methods for delivery of the devices of the present invention to the individual can be used and are known to the skilled artisan. Selection of the delivery method will depend upon the active pharmaceutical ingredient to be delivered and the shape of the device. For example, a vaginal ring-shaped delivery device can be administered by insertion of the delivery device into the vaginal lumen; a rod-shaped delivery device is administered by insertion subcutaneously; and a film-shaped delivery device is administered, e.g., orally, rectally or nasally via placement in oral, rectal or nasal cavity of the subject.

The polymeric outer shell and the API-loaded solid core can be manufactured by various means including, but not limited to hot melt extrusion, casting or any other molding process, such as injection molding.

Accordingly, the present invention also relates to methods for producing these drug delivery devices. The methods comprise forming a hollow polymeric outer shell having at least one closed internal cavity or compartment. The method further comprises inserting at least one solid core comprising one or more active hollow active pharmaceutical agents and one or more excipients into the closed internal cavity or compartment while maintaining an interspatial gap between the hollow polymeric outer shell and the solid core of the drug delivery device and forming the drug delivery device from the filled hollow polymeric outer shell and at least one solid core. In one nonlimiting embodiment, the hollow polymeric outer shell and/or the solid core are prepared by hot melt extrusion. In one nonlimiting embodiment, an agent is added to the hollow polymeric outer shell prior to or after inserting the at least one solid core. In one nonlimiting embodiment, the agent is an osmotic agent such as sodium chloride which promotes transfer of a biological fluid into the gap.

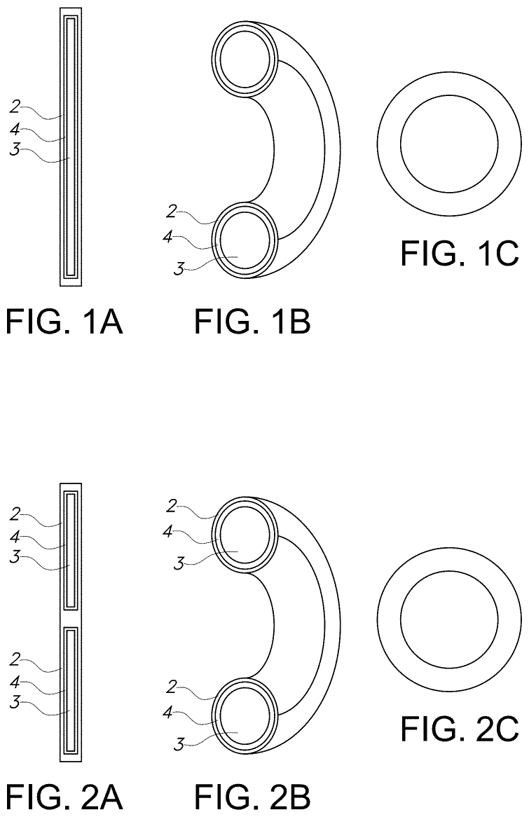

A nonlimiting embodiment of a drug delivery device of the present invention comprising a single compartmentalized vaginal ring is depicted in FIGS. 1A-1C. A nonlimiting embodiment of a drug delivery device of the present invention comprising a multi-compartmentalized vaginal ring is depicted in FIGS. 2A-2C. These FIGs. depict the device prior to bonding into a ring (FIG. 1A, FIG. 2A), a cross section of the ring (FIG. 1B, FIG. 2B), and an inner view of the complete ring (FIG. 10, FIG. 2C) and show the hollow polymeric outer shell 2 and the solid core 3 with the interspatial gap 4 in between.

Nonlimiting embodiments of devices of the present invention comprising a compartmentalized intravaginal ring were evaluated for the delivery of progesterone (PRG) as a model API. In these devices, the hollow polymeric outer shell of the device was comprised of a polyurethane (PU) and the solid core was comprised of a combination of PU and the API.

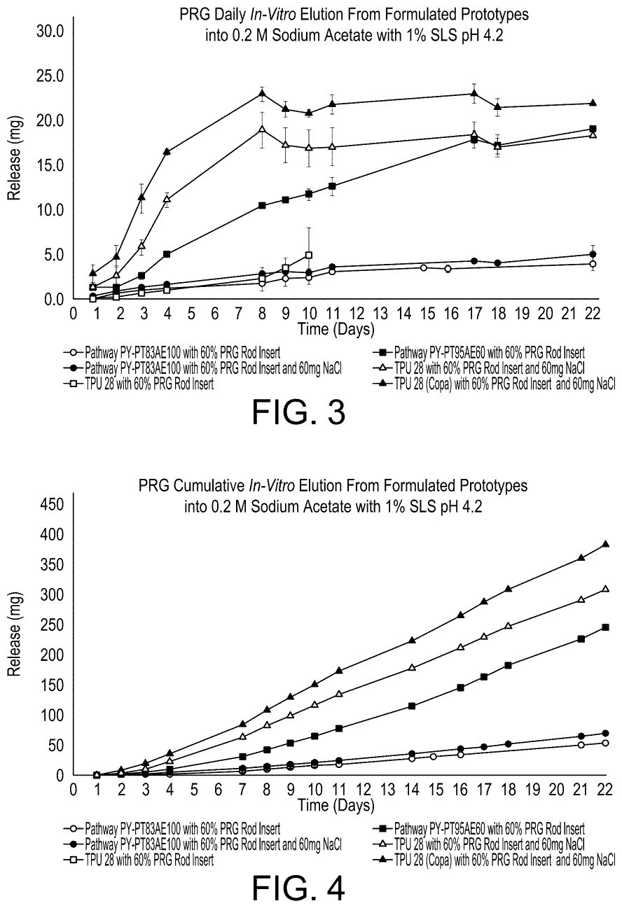

Experiments verified drug delivery using the devices of the present invention. Daily release from compartmentalized vaginal rings containing 60% PRG loaded solid cores in a polyurethane shell is depicted in FIG. 3 while cumulative PRG release from these devices is depicted in FIG. 4.

Further, it was demonstrated that release can be modified based on polymer properties and added agents.

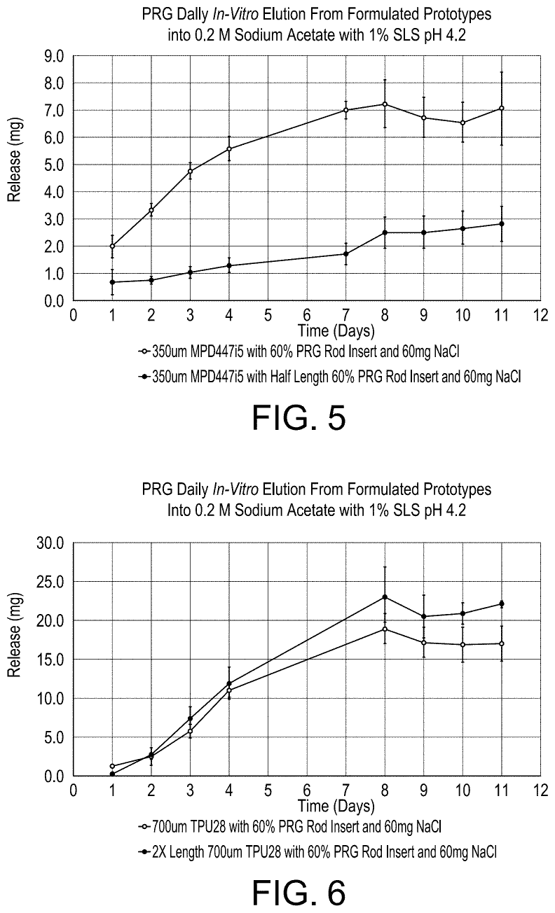

As shown in FIG. 5, daily release of progesterone from the formulated compartmentalized vaginal rings was higher with increased surface area of the solid drug contained core, thus demonstrating that release of a drug from a device of the present invention is dependent on the surface area of the solid core. Such control is useful, for example, in patient specific dosing with a subcutaneous implant where the trocar requires a fixed implant diameter for proper implantation. In this situation, the size of the solid core can be adjusted to provide the targeted daily drug dosing without modifying the overall implant size.

FIG. 6 shows results from experiments comparing changes in surface area of the hollow polymer shell on the daily release of progesterone from formulated compartmentalized devices of the present invention. Drug release was observed to be higher with increased surface area of the hollow polymer shell thus demonstrating that release of a drug from a device of the present invention is also dependent on the surface area of the hollow outer shell.

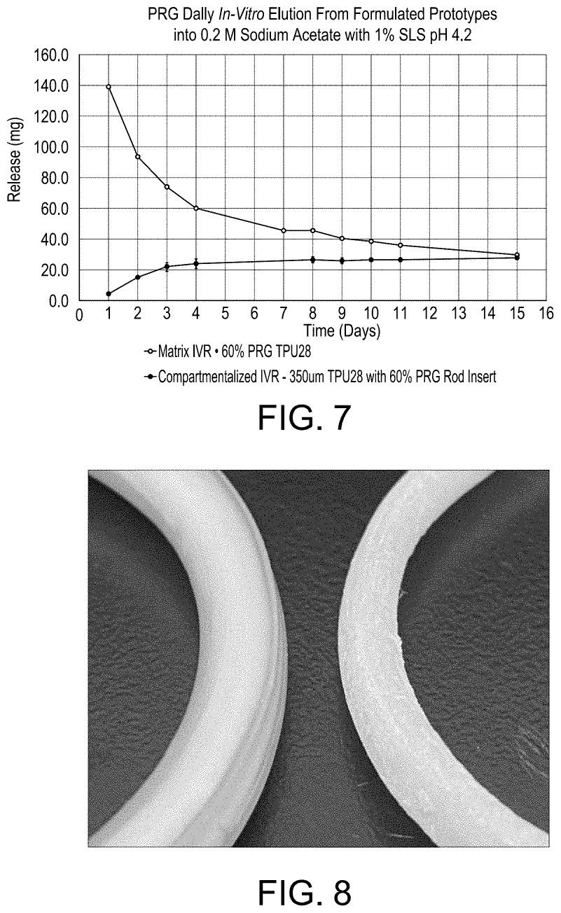

It has also been demonstrated that the compartmentalized design of the present invention is useful in controlling or dampening the release of a drug at the early timepoints, commonly referred to as a ‘Burst’. This burst is typically observed in conventional device designs (matrix and core-sheath), especially with high drug concentrations, where drug at the surface dissolves quickly into the surrounding fluid. FIG. 7 shows the release of the drug progesterone from a monolithic (Matrix) vaginal ring with the expected burst initially and a compartmentalized vaginal ring prepared in accordance with the present invention controlling or dampening the release of a drug at the early timepoints. Both rings were made with the same polymers and drug.

Further, unlike conventional matrix or reservoir (core-sheath) devices that have been well-studied, the compartmentalized device design is expected to release drug at a relative steady state even after the majority of the drug is depleted, as the drug concentration in the fluid that infiltrates the lumen of the ring during use is kept constant due to continuous dissolution of drug from the core replacing the eluted API. This steady state concentration allows the development of drug devices with minimum excess drug hence improving device safety and cost.

In addition, compartmentalized devices containing different amount of a drug in the solid core release the drug at similar rates. Thus, if the amount of drug remaining in a device of the present invention is higher, a longer duration of release will occur. Accordingly, devices of the present invention with higher drug loading will release drug at the same rate for a longer duration before the drug is depleted.

The devices of the present invention also prevent surface blooming of API during storage. FIG. 8 is a photograph comparing a compartmentalized device of the present invention (left) and a conventional core-sheath device (right) made with the same polymers and drug and aged for 14 months protected from light and moisture at ambient temperatures. The powdery substance on the surface of the core-sheath device is indicative of migration (blooming) of the drug progesterone to the surface of the ring, while no blooming was evident for the compartmentalized device, despite a much higher drug loading (60% vs 25%). This is useful to ensure stability of a drug device upon storage and reduces the risk of unintended drug exposure or transfer to a person in contact with the device.

The following nonlimiting examples are provided to further illustrate the present invention.

### EXAMPLES

### Example 1: Polymer Selection

Polymers as listed in Table 1 were selected for evaluation based upon hydrophilicity and hardness.

TABLE 1
Select Polymers Evaluated as Tubing

| | Water/Media | |
|---|---|---|
| Polymer | Absorption | Shore Hardness |
| Pathway ™ PY-PT83AE100 | ~100% | 83A |
| Pathway ™ PY-PT95AE60 | ~60% | 95A |
| MPD-447i (also referred to | ~35% | 85A (approx.) |
| as TPU28) | | |
| MPD-447ZA (also referred to | ~35% | 85A (approx.) |
| as TPU28 Copa)) | | |

### Example 2: Polymer Milling

To facilitate blending an active pharmaceutical ingredient with polymers and other excipients prior to production of the hollow polymeric shell and/or core, polymers were milled to a powder using a Retsch™ ZM200 Ultra Centrifugal Mill with a 750 μm distance sieve at a speed of 18,000 rpm. The use of liquid nitrogen or dry ice was required to prevent heat generation in the mill during the milling process. The polymer and liquid nitrogen, or dry ice, were fed into the mill concurrently and the collection vessel emptied as necessary.

### Example 3: Polymer Drying

Prior to use, polyurethanes were dried in a Dri-Air™ Industries NAFM Polymer Dryer in accordance with manufacturer recommendations. As typical drying time is greater than 4 hours, in most cases polyurethanes were dried overnight for use the next day. At the end of drying, dew points of approximately −45° F. were observed.

### Example 4: Powder Blending

To achieve a more homogeneous product and to simplify the feeding process during HME, a pre-extrusion powder blending was carried out in a Glen Mills T2F Turbula® Mixer. Milled TPU28 (Copa) polymer (40% w/w) and PRG (60% w/w) were serially mixed by manually mixing an approximately 1:1 ratio of PU and API, followed by sequential addition of API and additional mixing until the target batch size was achieved. The total batch was mixed for ten minutes at 46 rpm in the Turbula® mixer. A two-liter glass jar was used for mixing approximately 400-600 gram batches as necessary.

### Example 5: Compound Extrusion

A hot melt extrusion (HME) process using a Leistritz ZSE 18™ twin screw extruder was used for making compounds. Pre-mixed polymer and API blends were fed into the extruder with the aid of a Retsch® DR-100 vibratory feeder with a v-shaped chute attachment. The extruded material was drawn down to the desired diameter with a conveyor belt while being cooled with a series of Exair Super Air Knives™ and then the extrudate was pelletized with a Bay Plastics BT-25 pelletizer. Compounding parameters can be found in Table 2.

TABLE 2
Compounding Parameters

| | Z1 (° C.) | Z2 (° C.) | Z3 (° C.) | Z4(° C.) | Z5 (° C.) | Z6 (° C.) | Z7 (° C.) | Z8 (° C.) |
|---|---|---|---|---|---|---|---|---|
| Extrusion | 70 | 110 | 110 | 110 | 110 | 112 | N/A | 112 |
| Temps | | | | | | | | |

| Melt | Melt | | | |
|---|---|---|---|---|
| Temperature | Pressure | Screw Speed | Extruder | Cooling Water |
| (° C.) | (psi) | (rpm) | Load (%) | Temperature (° C.) |
| 113 | Below 200 | 130 | ~25-30 | 65 |

| | | | | | Air Cooling |
|---|---|---|---|---|---|
| Conveyor | Vibratory | Vibratory | Pelletizer | Pelletizer | Pressure |
| Speed (ftm) | Feeder Height | Feeder Speed | Pull Speed | Cut Speed | (psi) |
| 4-6.5 | N/A | N/A | 30 | 55 | ~40 |

### Example 6: Insert Extrusion

Extruded and pelletized PU/PRG compound was re-extruded by flood feeding through a ¾″ single screw extruder attached to a Brabender® ATR to form a solid rod of PU/PRG to be used as the tube insert. The PU/PRG rod was drawn down to the desired outer diameter (OD) with a Conair Medline puller/cutter and cut manually to the desired length. Insert extrusion parameters can be found in Table 3.

TABLE 3
Rod Extrusion Parameters

| Core Extruder | Z1 | Z2 | Z3 | Temp. Controller | | Speed | Screw | Feed Zone |
|---|---|---|---|---|---|---|---|---|
| Parameters | (° C.) | (° C.) | (° C.) | Z4 (° C.) | Z5 (° C.) | (RPM) | Type | Cooling |
| Set | 75 | 100 | 100 | 108 | 108 | 3 | Standard | None |
| Actual | 75 | 100 | 100 | 108 | 108 | 3 | Volume | |

| | | | | | |
|---|---|---|---|---|---|
| Average Torque (Nm) | ~45 | Avg. Pressure (psi) | 900-1000 | Melt Temp (° C.) | 99 |

### Example 7: Shell Extrusion

Polyurethane shells shaped as tubes were made by flood feeding polymer pellets through a ¾″ single screw extruder attached to a Brabender® ATR and passing the molten material through a Guill 812 tubing crosshead, Tip and dies were selected to produce a tube with a wall thickness of 0.70 mm and a 5.5 mm OD. Extruded tubes were passed through a Randcastle water trough to cool and drawn down to the desired OD with a Conair Medline puller/cutter. Additional tube dimensions of 5.5 mm OD with both 0.15 mm and 0.35 mm wall thicknesses were also made. Process parameters used in the tube manufacturing are detailed in Table 4 through Table 8. Tube wall thickness measurements are detailed in Table 9.

TABLE 4
Extrusion Parameters for 5.5 mm Pathway ™ PY-PT83AE100 Tubing (0.70 mm Wall)

| Extruder | Z1 | Z2 | Z3 | Temp. Controller | | Speed | Screw | Feed Zone |
|---|---|---|---|---|---|---|---|---|
| Parameters | (° C.) | (° C.) | (° C.) | Z4 (° C.) | Z5 (° C.) | (RPM) | Type | Cooling |
| Set | 140 | 155 | 160 | 160 | 140 | 15 | Std Vol | Air |
| Actual | 140 | 155 | 160 | 160 | 140 | 15 | | |

| | | | | | |
|---|---|---|---|---|---|
| Average Torque (Nm) | 44 | Avg. Pressure (psi) | 4090 | Melt Temp (° C.) | 152 |

| Cooling | | | | | | |
|---|---|---|---|---|---|---|
| | | Water Bath | Draw Down and Cutting | | | |
| | Air Cooling | Distance | Puller | Cut | Measured | Measured |
| | Pressure | from Die | Speed | Length | Strand OD | Strand Length |
| Cooling | (psi) | (cm) | (fpm) | (inch) | (mm) | (mm) |
| Water | N/A | 18.5 | 2.85 | 6.75 | 5.5 | 172 |

It was observed that tubing shrank about 2 mm in length after manufacturing. Therefore subsequent tubing was cut longer than the desired length to allow for shrinkage, and then cut to the desired length as necessary.

TABLE 5
Extrusion Parameters for 5.5 mm MPD447i Tubing (0.70 mm Wall)

| Extruder | Z1 | Z2 | Z3 | Temp. Controller | | Speed | Screw | Feed Zone |
|---|---|---|---|---|---|---|---|---|
| Parameters | (° C.) | (° C.) | (° C.) | Z4 (° C.) | Z5 (° C.) | (RPM) | Type | Cooling |
| Set | 138 | 143 | 149 | 147 | 147 | 10 | Std Vol | Air |
| Actual | 138 | 143 | 149 | 147 | 147 | 10 | | |

| | | | | | |
|---|---|---|---|---|---|
| Average Torque (Nm) | 3.8 | Avg. Pressure (psi) | 325 | Melt Temp (° C.) | 145 |

| Cooling | | | | | | |
|---|---|---|---|---|---|---|
| | | Water Bath | Draw Down and Cutting | | | |
| | Air Cooling | Distance | Puller | Cut | Measured | Measured |
| | Pressure | from Die | Speed | Length | Strand OD | Strand Length |
| Cooling | (psi) | (cm) | (fpm) | (inch) | (mm) | (mm) |
| Water | N/A | 7.5 | 1.9 | 7.1 | 5.4 | 180 |

TABLE 6
Extrusion Parameters for 5.5 mm MPD447ZA Tubing (0.70 mm Wall)

| Extruder | Z1 | Z2 | Z3 | Temp. Controller | | Speed | Screw | Feed Zone |
|---|---|---|---|---|---|---|---|---|
| Parameters | (° C.) | (° C.) | (° C.) | Z4 (° C.) | Z5 (° C.) | (RPM) | Type | Cooling |
| Set | 138 | 143 | 149 | 152 | 152 | 10 | Std Vol | Air |
| Actual | 138 | 143 | 149 | 152 | 152 | 10 | | |

| | | | | | |
|---|---|---|---|---|---|
| Average Torque (Nm) | 8.6 | Avg. Pressure (psi) | 455-475 | Melt Temp (° C.) | 147 |

| Cooling | | | | | | |
|---|---|---|---|---|---|---|
| | | Water Bath | Draw Down and Cutting | | | |
| | Air Cooling | Distance | Puller | Cut | Measured | Measured |
| | Pressure | from Die | Speed | Length | Strand OD | Strand Length |
| Cooling | (psi) | (cm) | (fpm) | (inch) | (mm) | (mm) |
| Water | N/A | 7.5 | 2.63 | 7.1 | 5.45 | 180 |

TABLE 7
Extrusion Parameters for 5.5 mm MPD447i Tubing (0.35 mm Wall)

| Extruder | Z1 | Z2 | Z3 | Temp. Controller | | Speed | Screw | Feed Zone |
|---|---|---|---|---|---|---|---|---|
| Parameters | (° C.) | (° C.) | (° C.) | Z4 (° C.) | Z5 (° C.) | (RPM) | Type | Cooling |
| Set | 138 | 149 | 149 | 147 | 147 | 10 | Std Vol | Air |
| Actual | 138 | 149 | 149 | 147 | 147 | 10 | | |

| | | | | | |
|---|---|---|---|---|---|
| Average Torque (Nm) | 3.5 | Avg. Pressure (psi) | 355 | Melt Temp (° C.) | N/R |

| Cooling | | | | | | |
|---|---|---|---|---|---|---|
| | | Water Bath | Draw Down and Cutting | | | |
| | Air Cooling | Distance | Puller | Cut | Measured | Measured |
| | Pressure | from Die | Speed | Length | Strand OD | Strand Length |
| Cooling | (psi) | (cm) | (fpm) | (inch) | (mm) | (mm) |
| Water | N/A | 8.5 | 2.63 | 7.5 | ~5.4 | 190 |

TABLE 8
Extrusion Parameters for 5.5 mm MPD447i Tubing (0.15 mm Wall)

| Extruder | Z1 | Z2 | Z3 | Temp. Controller | | Speed | Screw | Feed Zone |
|---|---|---|---|---|---|---|---|---|
| Parameters | (° C.) | (° C.) | (° C.) | Z4 (° C.) | Z5 (° C.) | (RPM) | Type | Cooling |
| Set | 138 | 149 | 149 | 147 | 147 | 10 | Std Vol | Air |
| Actual | 138 | 149 | 149 | 147 | 147 | 10 | | |

| | | | | | |
|---|---|---|---|---|---|
| Average Torque (Nm) | 3.8 | Avg. Pressure (psi) | 500 | Melt Temp (° C.) | N/R |

| Cooling | | | | | | |
|---|---|---|---|---|---|---|
| | | Water Bath | Draw Down and Cutting | | | |
| | Air Cooling | Distance | Puller | Cut | Measured | Measured |
| | Pressure | from Die | Speed | Length | Strand OD | Strand Length |
| Cooling | (psi) | (cm) | (fpm) | (inch) | (mm) | (mm) |
| Water | N/A | ~7.5 | 3.95 | N/A | N/A | N/A |

The 0.15 mm wall thickness tubing could not be manufactured consistently without the tubing collapsing on itself, likely due to the OD of the tube and the very thin wall. This led to a flatter profile than desired, which could not be passed through the cutter bushings of the puller/cutter. Therefore, tubing was collected in a long spool and manually cut to the desired length.

The average wall thickness of the various tubes used in the study can be found in Table 9.

TABLE 9
Tubing Wall Thickness Measurements

| Polymer | PY-PT83AE100 | PY-PT95AE60 | MPD447i | MPD447ZA | MPD447i | MPD447i |
|---|---|---|---|---|---|---|
| Ref/Batch | RD4283-02.B | 0101700391 | RD4283-17.A | RD4283-17.B | RD4283-29.A | RD4283-29.B |
| Number | | | | | | |
| Average Wall | 0.72 ± 0.01 | 0.38 ± 0.02 | 0.74 ± 0.02 | 0.63 ± 0.03 | 0.38 ± 0.03 | 0.17 ± 0.02 |
| Thickness | | | | | | |
| (μm) | | | | | | |

### Example 8: Ring Manufacturing

An open end of the extruded polyurethane tubes was thermally sealed using a PlasticWeld Systems HPS-EM tipping machine. For formulations that included sodium chloride (NaCl); NaCl was first added to the inner cavity (lumen) of the tube before the placement of the PU/PRG rod insert. The opposite end of the tube was thermally sealed. The sealed tubes containing the PU/PRG inserts were then thermally bonded into the shape of a ring using a PlasticWeld Systems HPS-20 bonder. Rings were packaged in mylar foil pouches and the pouches sealed with a continuous band heat sealer.

Approximate tipping and bonding parameters are detailed in Table 10 and Table 11. Parameters were similar for all formulations, with minor modifications based on tip and bond observations.

TABLE 10
HPS-EM Tipper Parameters

| | |
|---|---|
| Heat (sec) | 12.0 |
| Pre-Heat (sec | 9.0 |
| Cool (sec) | 20.0 |
| Clamp (psi) | 80 |
| Feed (psi) | 25-30 |
| Power (%) | 58.5-60.0 |
| L-Stage (Hole) | 6 |
| R-Stage (Hole) | 7 |
| L-Micrometer (inch) | 0.50 |
| R-Micrometer (inch) | ~0.1 |

TABLE 11
HPS-20 Bonder Parameters

| | |
|---|---|
| Pre-Heat (sec) | 2.0 |
| Heat 1 (%/sec) | 55/35.0 |
| Heat 2 (%/sec) | 62/5.0 |
| Heat 2 (%/sec) | 29/10.0 |
| Soak (sec) | N/A |
| Collent Open Delay (sec) | N/A |
| Cooling (sec) | 20.0-30.0 |
| Flag (psi) | 80 |
| Feed (psi) | 25 |
| Spot Cooler | Open |

### Example 9: Formulations Evaluated

Descriptions of formulations evaluated are set forth in Table 12.

TABLE 12
Formulation Descriptions

| | Tube | | |
|---|---|---|---|
| | Wall | | |
| | Thick- | | |
| | ness | | |
| Description | (um) | Lot Number | FID # |
| Pathway ™ PY-PT83AE100 with 60% | 700 | RD4283-13.A | 6319 |
| PRG Rod Insert IVR | | | |
| Pathway ™ PY-PT95AE60 with 60% | 700 | RD4283-13.B | 6320 |
| PRG Rod Insert IVR | | | |
| Pathway ™ PY-PT83AE100 with 60% | 700 | RD4283-20.A | 6352 |
| PRG Rod Insert and 60 mg NaCl IVR | | | |
| MPD-447i with 60% PRG Rod Insert | 700 | RD4283-20.B | 6353 |
| and 60 mg NaCl IVR | | | |
| MPD-447i with 60% PRG Rod Insert | 700 | RD4283-20.C | 6354 |
| IVR | | | |
| MPD-447ZA with 60% PRG Rod | 700 | RD4283-20.D | 6355 |
| Insert and 60 mg NaCl IVR | | | |
| MPD-447i with 60% PRG Rod Insert | 350 | RD4283-31.A | 6439 |
| and 60 mg NaCl IVR | | | |
| MPD-447i with 60% PRG Rod Insert | 150 | RD4283-31.B | 6440 |
| and 60 mg NaCl IVR | | | |

### Example 10: In Vitro Elution (IVE)

In vitro elution studies were carried out on ring prototypes to evaluate PRG release. Rings were submerged in 100-200 ml of 0.2M sodium acetate buffer (pH 4.2) containing 1% SLS as a surfactant and incubated in an orbital shaker set at 37° C. and 60 rpm. Elution media was changed daily, excluding weekends and holidays, for approximately 21 days.

### Example 11: Effects of API-loaded Solid Core Surface Area on Daily Drug Release

Experiments were performed to examine the effect of the drug-loaded solid core surface area on daily drug release from a compartmentalized device.

Compartmentalized vaginal rings containing a solid core comprised of the steroid hormone Progesterone (PRG) and thermoplastic polyurethane (TPU) were manufactured and evaluated for daily drug release in vitro. The vaginal rings were made with the form factor of a toroid, with the hollow outer shape having a wall thickness of 0.35 mm, minor diameter of 5.5 mm and major diameter of 54 mm. The solid cores were made with the form factor of a rod, with either a surface area of approximately 1784 mm² or approximately 1452 mm².

The polymers evaluated are listed in Table 13.

TABLE 13
Select Polymers Evaluated as Tubing

| Polymer | Water/Media Absorption |
|---|---|
| MPD-447ZA (also referred to as | 35% (approx.) |
| TPU28 (Copa)) | |
| MPD-447i5 | 0% (approx.) |

The hollow outer shell, shaped as tubes, were made using the MPD-447i5. The polymer was dried in a Dri-Air Industrial NAFM dryer for a minimum of 4 hours. At the end of drying, dew points of approximately −45° F. were observed. The dried polymer was flood fed through a ¾″ single screw extruder attached to a Brabender® ATR and the molten material passed through a Guill 812 tubing crosshead. Tip and dies were selected to produce a tube with a wall thickness of 0.35 mm and a 5.5 mm outer diameter. Extruded tubes were passed through a Randcastle water trough to cool and drawn down to the desired OD with a Conair Medline puller/cutter. Tube wall thickness measurements are detailed in Table 14.

The solid cores, shaped as cylindrical rods, were made using TPU28 (Copa). The TPU28 (Copa) polymer was milled using liquid nitrogen and a Retsch® ZM200 Ultra Centrifugal mill. The milled polymer was dried in a Dri-Air Industrial NAFM dryer for a minimum of 4 hours. At the end of drying, dew points of approximately −45° F. were observed. The dried TPU28 (Copa) polymer (40% w/w) and PRG (60% w/w) were blended using a Glen Mills T2F Turbula® Mixer. The pre-mixed polymer and API blends were compounded using a Leistritz ZSE18 twin screw extruder, drawn down and cooled on a conveyor belt with ExAir Super Air knives and pelletized with a Bay Plastic BT-25 pelletizer. The pelletized PU/PRG compound was injection molded into the shape of a ring, with a minor diameter of 4 mm and a major diameter of 54 mm, using an AB-200 bench top injection molder. The rings were cut along the minor diameter and straightened to form solid cylindrical rods with a length of 140 mm. An aliquot of the cylindrical rods was cut in half, lengthwise, producing solid cores in the shape of a truncated cylinder, with a reduced surface area. Rod length measurements, and respective surface areas, are detailed in Table 14.

An open end of the extruded tube was thermally sealed using a PlasticWeld Systems HPS-EM tipping machine. Sodium chloride (NaCl) was first added to the hollow compartment of the tubes before placement of the PU/PRG solid cores. The opposite end of the tube was thermally sealed. The sealed tubes containing the PU/PRG solid cores were then thermally bonded into the shape of a ring using a PlasticWeld Systems HPS-20 bonder. Rings were packaged in mylar foil pouches and the pouches sealed with a continuous band heat sealer.

Descriptions of formulations evaluated are set forth in Table 14.

TABLE 14
Formulation Descriptions

| | Tube Wall | Rod | | |
|---|---|---|---|---|
| | Thickness | Length | Rod Surface | |
| Description | (mm) | (mm) | Area (mm²) | Lot Number |
| MPD-447i5 with 60% PRG | 0.35 | 140 | 1784 (approx.) | RD4283_56.A |
| Rod Insert (full) and 60 mg | | | | |
| NaCl IVR | | | | |
| MPD-447i5 with 60% PRG | 0.35 | 140 | 1452 (approx.) | RD4283_56.B |
| Rod Insert (Truncated) and | | | | |
| 60 mg NaCl IVR | | | | |

In vitro elution studies were carried out on ring prototypes to evaluate PRG release. Rings were submerged in 100-200 ml of 0.2M sodium acetate buffer (pH 4.2) containing 1% SLS as a surfactant and incubated in an orbital shaker set at 37° C. and 60 rpm. Elution media was changed daily, excluding weekends and holidays, for approximately 14 days.

These experiments showed daily release of the drug in a compartmentalized device was controlled by adjusting the surface area of the solid API-loaded core.

### Example 12: Effects of Hollow Outer Shell Surface Area on Daily Drug Release

Experiments were performed to examine the effect of the hollow outer shell surface area on daily drug release from a compartmentalized device of the present invention.

Compartmentalized devices containing a solid core comprised of the steroid hormone Progesterone (PRG) and TPU were manufactured and evaluated for daily drug release. The devices were made with the form factor of a rod, with the hollow outer shape having an overall diameter of 5.5 mm, wall thickness of 0.70 mm and lengths of 151 mm or 322 mm. The solid cores were identical in each device, made with the form factor of a rod with an overall diameter of 4.0 mm and length of 140 mm.

The polymers evaluated are listed in Table 15.

TABLE 15
Select Polymers Evaluated as Tubing

| | Water/Media | |
|---|---|---|
| Polymer | Absorption | Shore Hardness |
| MPD-447ZA (also referred to as | 35% (approx.) | 85A (approx.) |
| TPU28 (Copa)) | | |
| MPD-447i (also referred to as | 35% (approx.) | 85A (approx.) |
| TPU28) | | |

The hollow outer shell, shaped as tubes, were made using MPD-447i (TPU 28). The polymer was dried in a Dri-Air Industrial NAFM dryer for a minimum of 4 hours. At the end of drying, dew points of approximately −45° F. were observed. The dried polymer was flood fed through a ¾″ single screw extruder attached to a Brabender® ATR and the molten material passed through a Guill 812 tubing crosshead. Tip and dies were selected to produce a tube with a wall thickness of 0.70 mm and a 5.5 mm outer diameter. Extruded tubes were passed through a Randcastle water trough to cool and drawn down to the desired OD with a Conair Medline puller/cutter. Tube wall thickness measurements, lengths and respective surface areas are detailed in Table 16.

The solid core, shaped as cylindrical rods, were made using TPU28 (Copa). The TPU28 (Copa) polymer was milled using liquid nitrogen and a Retsch® ZM200 Ultra Centrifugal mill. The milled polymer was dried in a Dri-Air Industrial NAFM dryer for a minimum of 4 hours. At the end of drying, dew points of approximately −45° F. were observed. The dried TPU28 (Copa) polymer (40% w/w) and PRG (60% w/w) were blended using a Glen Mills T2F Turbula® Mixer. The pre-mixed polymer and API blends were compounded using a Leistritz ZSE18 twin screw extruder, drawn down and cooled on a conveyor belt with ExAir Super Air knives and pelletized with a Bay Plastic BT-25 pelletizer. The pelletized PU/PRG compound was injection molded into the shape of a ring, with a minor diameter of 4 mm and a major diameter of 54 mm, using an AB-200 bench top injection molder. The rings were cut along the minor diameter and straightened to form solid cylindrical rods with a length of 140 mm.

An open end of the extruded tube was thermally sealed using a PlasticWeld Systems HPS-EM tipping machine. Sodium chloride (NaCl) was first added to the hollow compartment of the tubes before placement of the PU/PRG solid core rods. The opposite end of the tube was thermally sealed.

Descriptions of formulations evaluated are set forth in Table 16.

TABLE 16
Formulation Descriptions

| | Core Insert | Tube | | Tube | |
|---|---|---|---|---|---|
| | Dimensions | Wall | Tube | Surface | |
| | Length/OD | Thickness | Length | Area | |
| Description | (mm) | (mm) | (mm) | (mm²) | Lot Number |
| MPD-447i with 60% | 140/4 | 0.70 | 151 | 2630 | RD4283-20.B |
| PRG Rod Insert and | | | | | |
| 60 mg NaCl IVR | | | | | |
| MPD-447i with 60% | 140/4 | 0.70 | 322 | 5585 | 200116_For_000 |
| PRG Rod Insert and | | | | | |
| 60 mg NaCl IVR | | | | | |

In vitro elution studies were carried out on device prototypes to evaluate PRG release. Devices were submerged in 100-200 ml of 0.2M sodium acetate buffer (pH 4.2) containing 1% SLS as a surfactant and incubated in an orbital shaker set at 37° C. and 60 rpm. Elution media was changed daily, excluding weekends and holidays, for approximately 14 days.

These experiments showed daily release of the drug in a compartmentalized device was controlled by adjusting the surface area of the hollow outer shell.

### Example 13: Surface Migration (Blooming) Evaluation

Blooming evaluation was carried out by visually observing the surfaces of the rings; during storage at ambient conditions, for any API precipitation.

1. A delivery device for one or more active pharmaceutical agents, said device comprising:
a hollow polymeric outer shell forming at least one closed internal cavity or compartment and having an inner and outer surface; and
at least one solid core comprising one or more active pharmaceutical agents and one or more excipients inside said closed internal cavity or compartment and substantially unattached from said inner surface of said hollow polymeric outer shell of said device.

2. The delivery device of claim 1 wherein at least one solid core comprises one or more active pharmaceutical agents and a polymer.

3. The delivery device of claim 1 further comprising one or more agents in an interspatial gap.

4. The delivery device of claim 1 wherein the hollow polymeric outer shell comprises polyurethane.

5. The delivery device of claim 2 wherein at least solid core comprises polyurethane.

6. A method for producing a drug delivery device, said method comprising:
forming a hollow polymeric outer shell having at least one closed internal cavity or compartment;
inserting into the at least one closed internal cavity or compartment at least one solid core comprising one or more active pharmaceutical agents and one or more excipients while maintaining an interspatial gap between the hollow polymeric outer shell and the at least one solid core of the drug delivery device; and
forming the filled polymeric shell and solid core into the drug delivery device.

7. The method of claim 6 wherein the forming of the hollow polymeric outer shell is comprises forming the hollow polymeric outer shell by hot-melt extrusion, casting or other molding processes.

8. The method of claim 6 where the forming of the solid core comprises forming the solid core by hot melt extrusion, casting or other molding processes.

9. The method of claim 6 wherein the filled polymeric shell and solid core into the drug delivery device comprises forming the filled polymeric shell and solid core into a vaginal ring, a rod, a film or a patch.

10. The method of claim 6 further comprising adding an agent into the hollow polymeric outer shell prior to or after inserting the at least one solid core.

11. A method for delivering one or more active pharmaceutical agents to an individual in need thereof, said method comprising administering to the individual the drug delivery device of claim 1.
